(19) United States
(12) Patent Application Publication
(10) Pub. No.: US 2024/0041443 A1
(43) Pub. Date: Feb. 8, 2024

(54) Expandable plug

(71) Applicant: Stichting Radboud universitair medisch centrum, Nijmegen (NL)

(72) Inventors: Wilhelmina Francisca Daamen, Afferden (NL); Rob Thomas Christiaan Meuwese, Gendt (NL); Antonius H. M. S. M. van Kuppevelt, Nijmegen (NL)

(73) Assignee: Stichting Radboud universitair medisch centrum, Nijmegen (NL)

(21) Appl. No.: 18/258,814

(22) PCT Filed: Dec. 30, 2021
(86) PCT No.: PCT/EP2021/087883

§ 371 Date: Jun. 22, 2023

(30) Foreign Application Priority Data
Dec. 30, 2020 (EP) 20217909.9

(51) Int. Cl. A61B 17/00

(52) U.S. Cl. CPC A61B 17/0057; A61B 2017/00867; A61B 2017/00893; A61B 2017/00004

(57) ABSTRACT

The invention relates to the field of medicine. More specifically, the invention relates to an expandable plug for closing a defect in an organ or tissue without the need for sutures, wherein the plug has shape memory. The invention further relates to medical use of such plug.

### FIELD OF THE INVENTION

The invention relates to the field of medicine. More specifically, the invention relates to an expandable plug for closing a defect in an organ or tissue without the need for sutures, wherein the plug has shape memory. The invention further relates to medical use of such plug.

### BACKGROUND OF THE INVENTION

Many medical interventions require the puncturing of membranes. After being punctured, membranes can heal, but the puncture wound poses a risk until then. For instance, minimal fetal surgery using endoscopy is a promising medical technology to treat children before birth, e.g. children with rare diseases like congenital diaphragmatic hernia, twin-to-twin transfusion syndrome or spina bifida. During endoscopy, the maternal abdominal wall, uterus, and membranes surrounding the fetus are punctured. At the end of the procedure, the maternal wall and uterus are typically sutured, however, the punctured fetal membrane cannot be closed afterwards. As fetal membrane defects do not heal spontaneously (Gratacós et al. Placenta 2006; 27(4-5):452-456), this introduces a risk for leakage of amniotic fluid and a risk of up to 40% of membrane rupture.

Ruptures induced by fetal endoscopic surgery (also known as iatrogenic preterm premature rupture of membranes or iPPROM) are a strong trigger for premature birth, infection and maternal sepsis and are considered the “Achilles heel” of fetal endoscopic surgery as they can decrease or even annihilate the positive effects of fetal surgery. There is a need for beneficial interventions.

Multiple potential interventions have been suggested to reduce the iPPROM rate, but none have a proven positive effect. Luks et al, Am J Obstet Gynecol 1999; 181:995-6 proposed a gelatin plug which is compressed in a cannula and placed in fetoscopy port sites. Papadopoulos et al, In vivo 2010; 24: 745-750 proposed to use amnion cells as such, or on a scaffold, to facilitate closure of the fetal membrane. Gratacós et al, Am J Obstet Gynecol 2000; 182:142-6 proposed sealing of fetoscopic access sites with a non-expandable, static collagen plug. Engels et al, Prenatal diagnosis 2013; 33, 162-167 aimed at sealing of a fetal membrane defect by a collagen plug imbued with fibrinogen and plasma. Papanna et al, Ultrasound Obstet Gynecol 2013; 42: 456-460 described that an absorbable gelatin plug did not prevent premature rupture of the fetal membrane after surgery. Liekens et al, 2008 Prenatal Diagnosis; 28: 503-507 described that enrichment of collagen plugs with platelets and amniotic cells increases cell proliferation in sealed fetal membrane defects. Engels et al, Prenatal Diagnosis 2018; 38: 99-105 reported on four different sealant techniques to seal defects in fetal membranes with varying results. Altogether, multiple potential interventions have been advocated, but without demonstrated effectivity.

Accordingly, there is a need for an effective means for closing a membrane defect in e.g. a fetoscopic setting. There is a need for improved sealing of membrane defects. There is a need for reducing leakage after membrane puncture. There is a need for improved healing after membrane sealing. There is a need for reduced likelihood of complications arising from membrane puncture.

### SUMMARY OF THE INVENTION

A first aspect of the invention relates to an expandable plug for closing a defect in an organ or tissue without the need for sutures, wherein the material forming the plug has shape memory. In some embodiments, an expandable plug according to the invention is such that the material forming the plug is biodegradable. In some embodiments, an expandable plug according to the invention is such that the material forming the plug comprises or consists of a fibrillary material, such as collagen, such as type I collagen. In some embodiments, an expandable plug according to the invention is such that the material forming the plug is crosslinked, preferably wherein the crosslinking is via the formation of covalent bonds between functional groups present in the material forming the plug. In some embodiments, an expandable plug according to the invention is such that the plug has homogenous density and content throughout the plug. In some embodiments, an expandable plug according to the invention is such that the defect is in a membrane, such as a fetal membrane. In some embodiments, an expandable plug according to the invention is such that the defect is an endoscopic entry point, preferably wherein the defect is an endoscopic entry point created during fetal surgery. In some embodiments, an expandable plug according to the invention is such that it has a length of at most about 10 cm along its longest axis, and/or it has a length of about at most 1 cm along its shortest axis, preferably it has one longest axis and two substantially identical shorter axes.

Another aspect of the invention relates to an expandable made of a material having shape memory, for use as a medicament. In some embodiments, an expandable plug for use according to the invention is for closing a defect in an organ or tissue without the need for sutures, preferably wherein the defect is an endoscopic entry point, preferably an endoscopic entry point created during fetal surgery. Another aspect of the invention relates to an endoscopic device comprising a plug according to the invention. Another aspect of the invention relates to a method for loading a plug in an endoscopic device, the method comprising the step of inserting a plug according to the first aspect of the invention in the endoscopic device.

### DETAILED DESCRIPTION OF THE INVENTION

It has been established by the inventors that, surprisingly, a plug with shape-memory can close a membrane defect by directly expanding when it comes into contact with body fluid, hence overcoming the problems and needs as discussed herein. The plug can for instance be a collagen plug with shape memory.

Shape memory can be imparted to a scaffold such as a scaffold made of a fibrillary compound, such as type I collagen. Shape memory can be referred to as shape recovery. The plug can conveniently be used to close a defect in an organ or tissue, such as in a membrane, such as in a fetal membrane. Without being bound by the following theory, it is believed that by virtue of the shape memory, the plug will directly expand after placing and coming into contact with a polar fluid, such as amniotic fluid, will fixate itself in the membrane without the need for sutures, thereby closing the defect and preventing leakage of amniotic fluid.

Expandable Plug, Medical Methods and Uses

In a first aspect, there is provided an expandable plug made from a material having shape memory. Such plug is suitable for closing a defect in an organ or tissue without the need for sutures. Accordingly, in some embodiments, there is provided an expandable plug for closing a defect in an organ or tissue without the need for sutures, wherein the material forming the plug has shape memory.

A plug as used herein may be understood to refer to a piece of material fitting into and filling or blocking up a defect in an organ or tissue. The term expandable as used herein may be understood to refer to the ability to increase in size. A skilled person will understand that an expandable plug can be in its expanded state, for instance when it is not under any pressure or not constrained in any way. An expandable plug can also be in a compressed state, for instance when it is constrained in a small container or in a cannula or in an endoscopic device, or when it has been compressed and has not been in contact with an expansion stimulus. When a compressed plug is inserted in a perforation, for instance in a tissue, the plug may expand towards its expanded state, thus filling the available space in the perforation.

Shape memory as used herein may refer to the ability to recover to an original shape from a deformed shape, such as a crimped or compressed shape, preferably in response to a certain expansion stimulus. This recovery is repeatable. In the context of embodiments described herein, a preferred expansion stimulus is contact with a polar fluid, such as an aqueous fluid, preferably a bodily fluid, more preferably amniotic fluid. As used herein, a polar fluid is preferably an aqueous fluid, preferably a bodily fluid, more preferably amniotic fluid. An aqueous fluid preferably comprises at least 70 vol.-% water, more preferably at last 80, 90, or 95 vol.-% water, most preferably at least 99 vol.-% water.

Accordingly, in some embodiments, the material forming the plug as described herein is able to return substantially to its original shape after it has been compressed or crimped, preferably in response to an expansion stimulus, such as upon contact with a polar fluid. The recovery can be repeated more than once, such as at least 2, 3, 4, 5, 6, 7, 8, 9, 10, 11, 12, 13, 14, 15, or 20. Or 25, or 30, or 40, or 50, or 100, or 250, or 500 times. The inventors found that the plugs according to the invention could recover their expanded state after repeated pinching for at least 20 times without damaging or permanently deforming the plug.

In some embodiments, the material forming the plug as described herein, when not in its expanded state already, is able to increase in size and/or partly or fully regain its original size after it has been compressed or crimped, preferably in response to an expansion stimulus, such as upon contact with a polar fluid. Preferably, the plug expands to or towards its expanded size. Unless specifically indicated otherwise, the size of a plug is preferably determined while saturated with polar fluid.

An increase in size as used herein may mean an increase in size of about 50%, 75%, 100%, 1.25-fold, 1.5-fold, 1.75-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 9-fold, or 10-fold. In some embodiments, an increase in size may be at least about 50%, 75%, 100%, 1.25-fold, 1.5-fold, 1.75-fold, 2-fold, 2.5-fold, 3-fold, 3.5-fold, 4-fold, 4.5-fold, 5-fold, 5.5-fold or 6-fold. In some embodiments, an increase in size may be between 75% and 6-fold, preferably between 100% and 5.5-fold, more preferably between 1.25-fold and 5-fold, even more preferably between 1.5-fold and 4.5-fold. Such an increase in size is preferably from a compressed state, wherein the compressed state preferably entails compression of the plug in its expanded state by about the same amount as the desired expansion. For instance, an increase in size of 2-fold is preferably from a state where the plug has been compressed from its expanded size by at least about 2-fold, i.e. its diameter has been halved, after which an increase in size of 2-fold would restore the diameter to its dimension prior to compression. A skilled person will understand that an expandable plug may expand beyond its expanded state if the dimensions of the expanded state were determined under substantially dry conditions, because the expanded plug may swell in the polar fluid. Preferably, compression and/or expansion relate to at least a change in size of the diameter of the plug when it is substantially cylindrical, or of the plane defined by its two smallest diameters. This expansion is expected to contribute most to the sealing of a defect.

In a preferred embodiment, an increase in size may be at least 2.5-fold, 3-fold or 3.5-fold, preferably at least 3.5 fold. In some embodiments, an increase in size may be between 2.5-fold and 6-fold, preferably between 3-fold and 5.5-fold, more preferable between 3.5-fold and 5-fold. Such good results are typically obtained when the material forming the plug is crosslinked, as described later herein. It is highly preferred that plugs are crosslinked.

In some embodiments, the material forming the plug as described herein is able to regain about 10%, 15%, 20%, 25%, 30%, 35%, 40%, 50%, 60%, 70%, 80%, 90%, 95%, 100%, 105%, 110%, 115%, 120%, 125%, 130%, 135%, or 140% of its original size. In some embodiments, the material forming the plug as described herein is able to regain at least 20%, 25%, 30%, 35%, 40%, 50%, 60%, 70%, 80%, 90%, 95%, 100%, 105%, 110% or 115% of its original size. In some embodiments, the material forming the plug as described herein is able to regain from about 20% to about 140%, from about 25% to about 130% or from about 30% to about 120% of its original size.

In a preferred embodiment, the material forming the plug as described herein is able to regain at least 80%, 90%, 95%, 100%, 105%, 110%, 115%, or 120%, preferably at least 95%, of its original size. In some embodiments, the material forming the plug as described herein is able to regain at least substantially its original size after it has been compressed or crimped. In some embodiments, the material forming the plug as described herein is able to regain more than substantially its original size, such as at least 105%, preferably at least 110%, more preferably at least 115% of its original size. When comparing an original size to a compressed or crimped size, the same plug with the same features should be used for both states. For instance when one of the states is of a crosslinked plug, the other state should be of that same crosslinked plug.

In some embodiments, the material forming the plug as described herein is able to achieve the increase in size and/or to regain partly or fully its original size within 5 seconds, 10 seconds, 15 seconds, 20 seconds, 30 seconds, 45 seconds, 60 seconds, 2 minutes, 3 minutes, 5 minutes, 10 minutes, 20 minutes, 30 minutes, or one hour after an expansion stimulus. In a preferred embodiment, the material forming the plug as described herein is able to increase least 1.5-fold, more preferably twofold or more in size within 10 seconds after an expansion stimulus. In a preferred embodiment, the material forming the plug as described herein is able to regain about 70%, preferably about 80%, more preferably about 90% or more of its original size within 30 seconds, after an expansion stimulus. Such good results are typically obtained when the material forming the plug is crosslinked, as described later herein.

The increase in size may be expressed in terms of volume, surface area, length, width, height, breadth, depth, diameter, or the like. Preferably, the increase in size is expressed in terms of a diameter. Sizes can easily be measured using any suitable method known in the art, for example by using a ruler and/or a calliper, as described in the experimental section. Increase in size is preferably anisotropic. The increase in size is from the compressed state towards the expanded state, assuming no barriers are encountered. In practice barriers will be encountered, for instance the organ or tissue that can suitably be plugged. A skilled person will appreciate that the rate or direction of size increase is not critical as long as the defect in the organ or tissue will be closed. In other embodiments the increase in size is isotropic.

In some embodiments, an expandable plug as described herein is such that the material forming the plug is biodegradable. This means that the material may be degraded and eliminated from the body in a period of time, such as within 1 week, 2 weeks, 3 weeks, 4 weeks, 1 month, 2 months, 6 months, or 12 months. Examples of suitable biodegradable materials are polypeptides such as collagen or elastin-like polypeptides or fibrin, of which collagen is preferred. A biodegradable expandable plug can be degraded by the body, which may be important for subsequent pregnancies.

In some embodiments, an expandable plug as described herein is such that the material forming the plug comprises or consists of a network, fibres, filaments and/or fibrils, i.e. the material forming the plug comprises or consists of a network-, fiber-, filament- and/or fibril-forming compound. Fibers may be understood to be micro to milli-scale structures that are significantly longer than they are wide. Filaments may be understood to be long chains of protein. Fibrils may be understood to be linear, rod-like biopolymers having diameters ranging from 10-100 nanometers. Fibers, filaments and/or fibrils may connect with each other to form a network.

In some embodiments, a material comprising or consisting of a network, fibres, filaments and/or fibrils as described herein may be a polymer, such as a homopolymer and/or a copolymer. In some embodiments, a polymer may be a biological polymer or a synthetic polymer, preferably a biological polymer.

A synthetic polymer as described herein may be selected from the group consisting of: polyurethanes, polyethers, polyesters, polyamides, polyvinylchlorides, and silicon.

A preferred polymer is a biological polymer, such as a polysaccharide, a polynucleotide, or a polypeptide. Suitable biological polymers are collagen, actin, elastin, fibrin, keratin, resilin, silk, cellulose, and amylose. Preferred biological polymers are polypeptides, such as collagen, actin, elastin, fibrin, keratin, resilin and silk, more preferably collagen, elastin and fibrin, even more preferably collagen or elastin, most preferably collagen. These biological polymers are also fibrillary materials as described elsewhere herein. It is understood that elastin-like polypeptides are to be considered as elastin in the context of this invention.

In a preferred embodiment, an expandable plug as described herein is such that the material forming the plug comprises or consists of a fibrillary material. Preferably the material comprises 60%, 70%, 80%, 90%, 95%, 96%, 97%, 98%, 99%, or 100% by weight of fibrillary material, more preferably at least 90%, most preferably at least 98%. Fibrillary materials are structural biological materials found in nearly all living organisms. Fibrillary materials contain fibrils, i.e. linear, rod-like biopolymers having diameters ranging from 10-100 nanometers. In some embodiments, an expandable plug according to the invention comprises or consists of a fibrillary material selected from the group consisting of: collagen, actin, elastin, fibrin, keratin, resilin, silk, cellulose and amylose. Preferably, an expandable plug as described herein comprises or consists of a protein-based fibrillary material, preferably selected from the group consisting of: collagen, actin, elastin, fibrin, keratin, resilin and silk, preferably selected from the group consisting of: collagen, elastin and fibrin, more preferably collagen or elastin. A particularly preferred fibrillary material is collagen, preferably type I collagen.

In the context of embodiments described herein, collagen may be medical grade collagen or non-medical grade collagen. Preferably, collagen is medical grade collagen. Suitable collagens include collagen types I, II, III, V and XI. A preferred collagen is type I collagen. A preferred type I collagen is medical grade type I collagen. Highly purified collagen used for the plug will elicit minimal inflammatory response.

Type I collagen can stimulate migration of human amnion mesenchymal cells. The porosity of type I collagen allows cellular ingrowth from the surrounding tissue, which may further tighten the plug over time and stimulate regeneration of membrane tissue.

It is known that collagen can be denatured, for instance by exposing it to an alkaline treatment that increases the thickness of the material (see US 2009/0326577). This denaturation by alkaline treatment disrupts intramolecular and intermolecular bonds in the native collagen. It was found that plugs formed of denatured material had inferior shape memory. Accordingly, preferably the material forming plugs according to the invention comprises or consists of native collagen. As used herein, native collagen is collagen that has not been denatured by an alkaline treatment. An alkaline treatment can for instance be soaking of the material in at least 1 M or 2 M NaOH in water for prolonged periods of time, for instance 2 or more hours, for instance at 37° C. A preferred type of collagen is thus native collagen. A preferred type of collagen is thus collagen that has not been denatured by an alkaline treatment. It was found that native collagen allowed for the formation of plugs that could recover from deformation after swelling. Such plugs are desirable for clinical applications, for instance because a subject is mobile. A preferred type of native collagen is native type I collagen. A preferred type of native collagen is crosslinked native collagen. Most preferred is crosslinked native type I collagen.

In some embodiments, the material forming the expandable plug as described herein is crosslinked. By crosslinking, the material such as the fibrillary compound can be brought into an energetically favourable form to allow and/or to improve shape memory. It is expected that crosslinking while the object is in a given shape can render that shape more energetically favourable, improving memory towards that shape from other shapes. Crosslinking is known in the art. Generally, a crosslinked material has bonds between otherwise distinct elements of that material. In the present case, polymers in the plug can be linked to one another via crosslinking, forming a network.

Particularly when the material is crosslinked, it is able to increase in size after it has been compressed or crimped, preferably in response to an expansion stimulus, such as upon contact with a polar fluid. An increase in size may be at least 2.5-fold, 3-fold or 3.5-fold, preferably at least 3.5 fold. In some embodiments, an increase in size may be between 2.5-fold and 6-fold, preferably between 3-fold and 5.5-fold, more preferable between 3.5-fold and 5-fold. Put differently, particularly when the material is crosslinked, it is able to regain at least 80%, 90%, 95%, 100%, 105%, 110% or 115%, preferably at least 95%, of its original size after having been crimped or compressed. In some embodiments, it is able to regain at least substantially its original size after it has been compressed or crimped. In some embodiments, it is able to regain more than substantially its original size, such as at least 105%, preferably at least 110%, more preferably at least 115% of its original size.

In a preferred embodiment, the crosslinking is via the formation of covalent bonds between functional groups present in the material forming the plug. In some embodiments, the crosslinked material may be obtained by a chemical, physical or biological crosslinking method, or any combination thereof, preferably a chemical crosslinking method.

Suitable physical crosslinking methods are crosslinking induced by temperature (dehydrothermal crosslinking), pressure mediated crosslinking, pH mediated crosslinking, radical mediated crosslinking, and irradiation mediated crosslinking such as UV irradiation mediated crosslinking. Suitable biological crosslinking methods are enzymatic crosslinking, such as enzymatic crosslinking induced by lysyl oxidase (LOX) or transglutaminase such as microbial transglutaminase (MTG). In a preferred embodiment, the material forming the expandable plug as described herein is chemically crosslinked. Chemical crosslinking may be induced by click chemistry or by one or more crosslinkers selected from: carbodiimides (such as 1-ethyl-3-(3-dimethyl aminopropyl)carbodiimide (EDC) or dicyclohexylcarbodiimide (DCC) or N,N′-diisopropylcarbodiimide (DIPCDI)), active esters such as imidazole esters (for instance as used in 1,1′-carbonyldiimidazole), N-hydroxysuccinimide esters (such as N-hydroxysuccinimide (NHS) for instance in disuccinimidyl suberate, sulfo-NHS for instance in bis(sulfosuccinimidyl) suberate), imidoesters (such as dimethyl suberimidate) and aldehydes (such as formaldehyde, glutaraldehyde, and dialdehyde starch). Preferably, chemical crosslinking may be induced by carbodiimide crosslinkers such as EDC and DCC, preferably EDC, or aldehyde crosslinkers such as formaldehyde, glutaraldehyde and dialdehyde starch. A most preferred chemical crosslinker is water soluble, such as EDC. In preferred embodiments carbodiimides can be used as crosslinkers in the presence of NHS. Methods for crosslinking using such reagents are widely known.

Chemical crosslinking can strengthen the plug and increase its degradation time, rendering the plug more stable while in use. This can be attractive to ensure the plug endures the further duration of pregnancy. In preferred embodiments the plug remains stable while exposed to a polar liquid for at least 2 weeks, preferably at least 4 weeks, more preferably at least 6 weeks, more preferably at least 10, 12, 14, 16, 18, 20, 22, 24, 26, 28, or 30 weeks, most preferably at least 24 weeks. Stability of a plug is preferably the ability to retain a good seal in the membrane defect. In general a seal can be considered good when the flow of liquids through the defect is reduced by at least 80%, preferably at least 95%, more preferably at least 99%, as compared to the flow through the defect prior to sealing. Preferably this flow is reduced by at least 90, 91, 92, 93, 94, 95, 96, 97, 98, 99, 99.5, 99.8, 99.9, or 100%. This can mean that the flow is halted entirely, which is most preferred. Fluid leakage can be detected as demonstrated in the examples.

The use of carbodiimides can be referred to as zero-length crosslinking, because no linker atoms are introduced. It was found that zero-length crosslinkers lead to plugs with good shape memory, while the use of linkers could lead to slower recovery of the original shape. Accordingly in preferred embodiments the crosslinker is a zero-length crosslinker.

A crosslinker is preferably present during crosslinking at a concentration of 1 mM to 100 mM, more preferably of about 10 mM to about 50 mM. In preferred embodiments, at least 20 mM crosslinker is present. In preferred embodiments, at most 40 mM, more preferably at most 35 mM, still more preferably about 33 mM crosslinker is present. Crosslinking is preferably performed for 0.25-10 hours, more preferably 0.5-6 hours, even more preferably 1-5 more preferably 1.5-4 hours, more preferably 2-3.5 hours, most preferably 2.5-3.5 hours such as 3 hours. Crosslinking is preferably performed at ambient temperatures.

When present, a linker is preferably present during crosslinking at a concentration of 1 μM to 10 mM, more preferably of about 1 μM to about 2 mM. In preferred embodiments, at least 2 μM linker is present. In preferred embodiments, at most 100 μM, more preferably at most 50 μM, still more preferably at most 10 μM, even more preferably at most 5 μM linker is present. In other embodiments a linker is present at a concentration of 0.5 to 10 mM, preferably 2 to 5 mM, more preferably 3 to 4 mM.

In other embodiments, when slower expansion is desired, at least 100 μM, more preferably at least 500 μM, still more preferably at least 750 μM, even more preferably at least 900 μM linker is present, such as about 1 mM linker. In these embodiments, preferably at most 100 mM, more preferably at most 10 mM, still more preferably at most 5 mM, even more preferably at most 2 mM linker is present.

In some embodiments, the material is crosslinked with the help of a linker. Such a linker should have at least two functional groups which can form a bond with complementary functional groups in the material forming the plug. In preferred embodiments the at least two functional groups are the same, resulting in a homobifunctional linker. For instance, a linker can have two amines. Such amines can form amide bonds with carboxylic acid groups present in the material forming the linker. Examples of linkers comprising two amines are lysine, ornithine, putrescine, cadaverine, and 1,6-diaminohexane. A linker can also have two carboxylic acids, for instance glutamate, aspartate, maleic acid, fumaric acid, and others. Other suitable linkers comprise both a carboxylic acid and an amine, such as 6-aminohexanoic acid. Use of a diamine linker consumes carboxylic acids in the material. Use of a dicarboxylic acid linker consumes amines in the material. Such linkers can influence the overall charge of the material. Preferred linkers are small and optionally have at most 35 atoms, more preferably at most 30 atoms. Preferred linkers result in a linker with a length of at most 15 atoms, preferably at most 10 atoms, more preferably at most 8 atoms, more preferably at most 6 atoms, such as 1,6-diaminohexane. Highly preferred linkers are 1,6-diaminohexane and adipic acid.

In some embodiments, the material forming the expandable plug as described herein is crosslinked, preferably chemically crosslinked, wherein the crosslinking method is zero-length crosslinking or spacer crosslinking. Zero-length crosslinkers achieve crosslinking without the introduction of a spacer. In a preferred embodiment, the material forming the expandable plug as described herein is chemically crosslinked using a zero-length crosslinker, such as a carbodiimide, preferably EDC or DCC or DIPCDI, more preferably EDC, optionally while using NHS.

Suitable crosslinking methods for collagen are known in the art, as described for example in Adamiak et al. Current methods of collagen cross-linking: a review. International Journal of Biological Macromolecules 2020; 116:550-560. Suitable chemically crosslinked forms of collagen as described herein are collagen crosslinked by glutaraldehyde, genipin, 1-ethyl-3-(3-dimethyl aminopropyl)carbodiimide (EDC) and N-hydroxysuccinimide (NHS), dialdehyde starch and chitosan.

A preferred method for providing a plug according to the invention comprises the steps of:
a) Providing a collagen source such as animal tendons, preferably bovine tendons;
b) Purifying collagen from the source to obtain native collagen, preferably without exposing the collagen to an alkaline denaturation treatment;
c) Swelling the native collagen in a suitable liquid such as aqueous acetic acid to obtain swollen collagen;
d) Casting the swollen collagen in a mould to obtain cast collagen and lyophilizing the cast collagen to form an expandable plug;
e) Crosslinking the expandable plug.

The steps of this method are preferably performed in alphabetical order. Collagen sources for step a) are widely known in the art. Purification of collagen from the source is also well known. As an example, collagen such as type I collagen can be isolated from tendons such as bovine tendons by pulverizing the tendons, followed by washing steps with aqueous solutions of NaCl, urea, acetic acid, acetone and demineralized water.

The swelling of step c) is advantageously performed by suspending the purified collagen fibrils in a suitable solution, such as aqueous acetic acid, for instance 0.1-5 M acetic acid in water, preferably 0.2-2 M acetic acid in water, more preferably 0.2-0.4 M acetic acid, such as about 0.25 M acetic acid in water. The suspension is preferably a 1.5 wt.-% collagen suspension, but can have any wt.-% range as indicated elsewhere herein. It can be 0.4-7 wt.-%, or 0.5-5 wt.-%, preferably 0.7-4 wt.-%, more preferably 0.8-3 wt.-%, more preferably 0.9-2.5 wt.-%, most preferably 1-2 wt.-%. Attractive suspensions comprise 1-1.5 wt.-% collagen. Swelling is preferably performed for at least 1, 2, 3, 4, 5, 6, 7, 8, 9, 10, 11, 12, 13, 14, 15, or 16 hours, such as for at least 4 or preferably at least 8 hours. Swelling overnight is convenient. Swelling is preferably not for longer than 240 hours, more preferably not longer than 72 hours. After swelling the suspension is preferably homogenized. Casting is conveniently done in a tube with the desired plug diameter, such as a tube with an inner diameter of 9.9 mm. Preferably the presence of air bubbles is reduced, for instance by careful casting. The filled tubes were can then be frozen to prepare for lyophilization. Freezing can be performed using liquid nitrogen or using storage at a sufficiently low temperature, such as at −196° C., −80° C. or −20° C., preferably −20° C. Lyophilization can be performed using any known suitable method.

Lyophilized plugs can be crosslinked as described above, preferably using a zero-length crosslinking method, for instance applying 1-ethyl-3-(3-dimethylaminopropyl)carbodiimide (EDC) and N-hydroxysuccinimide (NHS). For crosslinking, the plugs are preferably wetted such as wetted for at least 1, 2, 3, 4, 5, 6, 7, 8, 9, 10, 11, 12, 13, 14, 15, or 16 hours, such as for at least 4 or preferably at least 8 hours. Wetting overnight is convenient. Wetting is preferably not for longer than 240 hours, more preferably not longer than 72 hours. Wetting is preferably in a suitable aqueous buffer such as 50 mM 2-morpholinoethane sulfonic acid (MES buffer, pH 5.0) containing 40% (v/v) ethanol. Crosslinking is preferably performed in that buffer for a length of time as described elsewhere herein, preferably 3 h, preferably at ambient temperature, using a concentration of crosslinking agent as described elsewhere herein, such as using 33 mM EDC and 6 mM NHS in 50 mM MES buffer (pH 5.0), containing 40% (v/v) ethanol. After crosslinking the plug is preferably washed, such as washed with 0.1 M Na₂HPO₄, 1 M NaCl, 2 M NaCl, and demineralized water, after which plugs are preferably lyophilized again. Plugs can be optionally cut to a desired length as described elsewhere herein.

A useful additional step can be step g) crimping the crosslinked expandable plug. Crimping can be done using known crimping equipment. Preferably the plug is crimped to reduce at least one of its dimensions to at most 50% of its initial value. In preferred embodiments the crimping reduces to at most 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10% of the initial value. Reductions to at most 40%, preferably at most 35% of the initial value are useful for practical applications.

An expandable plug as described herein may have any suitable form or shape. In some embodiments, the plug is solid, i.e. non-hollow. While porous, such a plug does not have macroscopic cavities or empty spaces on its inside, and does not enclose an empty space. In some embodiments, the plug has homogenous density and content throughout the plug. A rolled-up sheet does not form a plug with homogenous density because the sheet can leave minute interstitial spaces and the rolling can lead to stresses and bulges that influence local density.

In some embodiments, an expandable plug as described herein has a maximal length of about 15, 14, 13, 12, 11, 10, 9, 8, 7, 6, 5, 4, 3, 2, or 1.5 cm, preferably about 10 cm, in any direction and/or a minimal length of 0.1, 0.2, 0.3, 0.4, 0.5, 0.6, 0.7, 0.8, 0.9, 1, 1.1, 1.2, 1.3, 1.4 or 1.5 cm, preferably about 1 cm, in any direction. In other preferred embodiments the smallest length in any direction is about 4 to 6 mm, such as about 4 mm. The maximal length in any direction may also be referred to as the length along the longest axis; the minimal length may also be referred to as the length along the shortest axis. Accordingly, in some embodiments, an expandable plug as described herein has a length of at most about 15, 14, 13, 12, 11, 10, 9, 8, 7, 6, 5, 4, 3, 2, or 1.5 cm, preferably about 10 cm, along its longest axis and/or a length of at most about of 0.1, 0.2, 0.3, 0.4, 0.5, 0.6, 0.7, 0.8, 0.9, 1, 1.1, 1.2, 1.3, 1.4 or 1.5 cm, preferably about 1 cm, along its shortest axis. Preferably, the expandable plug has one longest axis and two shorter axes, optionally the two shorter axes are substantially identical. It is highly preferred that the length of the shortest axis, which is preferably the length of both shortest axes, is about 1 cm, or is in the range of about 0.75 cm to 1.2 cm, referring to the expanded state of the plug. In other preferred embodiments the length of the shortest axis is about 0.1 cm to about 0.5 cm, or about 0.15 cm to about 0.45 cm, such as about 4 mm.

In some embodiments, an expandable plug as described herein has a maximal length in any direction (or length along its longest axis) of from about 1.5 to about 15 cm, from about 6 to about 14 cm, from about 7 to about 13 cm, from about 8 to about 12 cm, from about 9 to about 11 cm or of about 10 cm. In some embodiments, an expandable plug as described herein has a minimal length in any direction (or length along its shortest axis) of from about 0.1 to about 1.5 cm, from about 0.3 to about 1.4 cm, from about 0.5 to about 1.3 cm, from about 0.7 to about 1.2 cm, from about 0.9 to about 1.1 cm or of about 1 cm.

In some embodiments, an expandable plug as described herein is substantially longer than it is wide. An expandable plug as described herein may have a greater length in one direction and two shorter lengths in the remaining orthogonal directions, optionally the two shorter lengths are substantially the same. In other words, an expandable plug as described herein may have one longest axis and two shorter axes, optionally the two shortest axes are substantially identical. In some embodiments, the greater length (or the length along the longest axis) may be at most about 15, 14, 13, 12, 11, 10, 9, 8, 7, 6 or 5 cm, preferably about 10 cm, and/or both shorter lengths (or the lengths along the shortest axes) may be at most about of 0.1, 0.2, 0.3, 0.4, 0.5, 0.6, 0.7, 0.8, 0.9, 1, 1.1, 1.2, 1.3, 1.4 or 1.5 cm, preferably about 1 cm. Preferably, in some embodiments, the greater length (or the length along the longest axis) as described herein may be from about 5 to about 15 cm, from about 6 to about 14 cm, from about 7 to about 13 cm, from about 8 to about 12 cm, from about 9 to about 11 cm or about 10 cm and/or both shorter lengths (or the lengths along the shortest axes) as described herein may be at most about 0.5 to about 1.5 cm, from about 0.6 to about 1.4 cm, from about 0.7 to about 1.3 cm, from about 0.8 to about 1.2 cm, from about 0.9 to about 1.1 cm or of about 1 cm, or of from about 0.1 to about 1.5 cm, from about 0.3 to about 1.4 cm, from about 0.5 to about 1.3 cm, from about 0.7 to about 1.2 cm, from about 0.9 to about 1.1 cm or of about 1 cm.

A plug that is longer than the desired length along its longest axis can conveniently be cut to the desired length along its longest axis using any suitable cutting means, such as scissors or scalpels. Thus, an expandable plug as described herein may have a substantially rod-like shape, or it may be substantially cylindrical. Despite the length limits described herein, the plug may be produced with a substantially longer length and cut into smaller fragments prior to use.

In some embodiments, an expandable plug as described herein may comprise additional bioactive components, optionally selected from the group consisting of antibiotics, growth factors, and platelets, most preferably antibiotics. In preferred embodiments, the plug consists essentially of the material forming it, as described elsewhere herein.

A second aspect of the invention relates to the expandable plug as described herein, for use as a medicament. Such a plug for medical use is referred to herein as a plug for use according to the invention. In some embodiments, an expandable plug for use as described herein is for closing a defect in an organ or tissue without the need for sutures. In preferred embodiments the expandable plug for use is for treating or preventing iPPROM, preferably preventing iPPROM.

Also provided herein is a method for closing a defect in an organ or tissue without the need for sutures, the method comprising closing the defect in the organ or tissue by inserting therein an expandable plug as described herein.

In a preferred embodiment, the expandable plug may be inserted by using an endoscopic device. In a preferred embodiment, the endoscopic device is suitable for use during a fetoscopy procedure, i.e. it is a fetoscopic endoscope.

In some embodiments of an expandable plug for use, a method or a use as described herein, the use or method may be for closing a defect in an organ or tissue in a subject in need thereof.

In the context of expandable plugs for use, methods and uses of the invention, a defect may be a closable or sealable defect, such as a perforation, a cut, an incision, an opening, a hole, a penetration, a rupture, and the like. A preferred defect is a perforation, and a preferred perforation is an endoscopic entry point, preferably an endoscopic entry point generated during fetal surgery.

In the context of any embodiment herein, relating to expandable plugs, expandable plugs for use, methods and uses as described herein, an organ or tissue may be selected from the group consisting of: adrenal glands, anus, appendix, bladder, bones, bone marrow, brain, bronchi, diaphragm, ears, eyes, fallopian tubes, gallbladder, olfactory epithelium, heart, hypothalamus, joints, kidneys, large intestine, larynx, liver, lungs, lymph nodes, mammary glands, mesentery, mouth, nasal cavity, nose, ovary, pancreas, pineal gland, parathyroid glands, pharynx, pituitary gland, prostate, rectum, salivary glands, skeletal muscles, skin, small intestine, spinal cord, spleen, stomach, teeth, thymus gland, thyroid, trachea, tongue, ureters, urethra, uterus, nerves, ligaments, tendons, clitoris, vagina, vulva, cerebellum, placenta, testes, epididymis, vas deferens, seminal vesicles, bulbourethral glands, penis, scrotum, subcutaneous tissue, foramen ovale, arteries, veins, capillaries, lymphatic vessel, tonsils, and interstitium. More preferred organs or tissues are inside the body. In some embodiments, the defect is in an organ or tissue that is not the teeth or skin or ears.

In some embodiment, the expandable plugs and expandable plugs for use are for sealing dental defects. Such an expandable plug can fill a defect that is left after removal of a tooth, such as removal of a molar, premolar, canine, or incisor. The expandable plugs are suitable for absorbing blood and inducing coagulation, and can therefore be used directly after removal of the tooth. An advantage is that the plug will degrade after an amount of time, such as after 1, 2, 3, 4, 5, 6, 7, 8, 9, 10, 11, or 12 months. The plug thus promotes healing of the defect after removal of a tooth, obviating the need for placement of a dental implant. In other embodiments the expandable plug and the expandable plug for use is for sealing dental defects, absorbing blood, locally inducing blood coagulation, and inducing bone formation. Such an expandable plug is useful for when placement of an implant at a later stage is desired.

In some embodiments, the defect in an organ or tissue is selected from the group consisting of:
a perforation in an organ, preferably the intestines, including small intestine, large intestine and rectum, lung and/or bladder; in some embodiments the perforation is a fistula;
a lesion in a membrane, preferably peritoneum, dura mater, lung membrane and fetal membrane, more preferably fetal membrane; and
a hernia, preferably an umbilical hernia, an inguinal hernia, a femoral hernia, a spinal disc herniation, and optionally hernia nucleus pulposus;
a cardiovascular defect, preferably a patent foramen ovale, pseudoaneurysm and/or ventricular septum defect;
an endoscopic entry point, preferably an endoscopic entry point generated during fetal surgery; and
a macular lesion.

In a preferred embodiment the defect in an organ or tissue may be a defect in a membrane. A membrane may be understood herein as referring to a thin sheet of tissue or layer of cells acting as a boundary, lining, or partition in an organism. A membrane may be selected from the group consisting of: peritoneum, dura mater, lung membranes and fetal membranes. A preferred membrane is a fetal membrane.

In preferred embodiments, an expandable plug, expandable plug for use, a method or a use restores and/or creates a barrier function. The plug prevents leakage of fluids, preferably it prevents leakage of the fluid that is also the expansion trigger. Contact with that fluid incited expansion of the plug, causing it to tightly fill the defect in the tissue or organ, and preventing the further leakage of fluid.

Endoscopic Device

A third aspect of the invention relates to an endoscopic device comprising an expandable plug as described herein. Endoscopic devices are well known and are commercially available from various suppliers. An endoscope or endoscopic device comprises a long, thin, and generally flexible tube with an optional camera on its tip, such as a fiber-optic camera. It contains a working channel, meaning the tube is hollow, and it enables operators such as physicians to insert surgical instruments through the endoscope. An endoscopic device can advantageously insert a plug according to the invention in a defect in a tissue or organ. The endoscopic device is thus used for delivery or placement of the plug.

In a preferred embodiment, the endoscopic device is for use during a fetoscopy procedure, i.e. it is fetoscopic endoscope.

Methods

A further aspect of the invention relates to a method for loading a plug in an endoscopic device, the method comprising the step of inserting an expandable plug as described herein in the endoscopic device. In a preferred embodiment, the endoscopic device is for use during a fetoscopy procedure, i.e. it is fetoscopic endoscope. Preferably the plug is compressed or crimped before being loaded in the endoscopic device. In a preferred embodiment, the plug has an initial diameter that is larger than the diameter of the endoscopic device, and is compressed to a smaller diameter to allow the plug to fit inside the endoscopic device. It is convenient to use a compressed plug that has its diameter in compressed state substantially match the inner diameter of the endoscopic device.

A plug as disclosed herein can be produced using any method known to the person skilled in the art, such as by providing a suspension or a solution of the material, homogenizing the suspension or solution, casting, freezing and lyophilizing. Accordingly, in an aspect, there is provided a method of manufacturing an expandable plug as described herein, comprising:
providing a suspension or a solution of a material for forming the plug as described herein;
homogenizing the suspension or solution;
casting the suspension or solution;
freezing the suspension or solution, preferably at a temperature of about −196° C. to about −5° C., such as −78° C., or preferably −20° C.; and
optionally lyophilizing the suspension or solution,
   to form an expandable plug as described herein. Instead of casting the suspension, the plug can also be extruded or spun, for instance using electrospinning. Instead of lyophilizing, the plug can also be solidified using phase separation using a miscible organic liquid. Provision of a suspension of a material is preferably in a liquid suitable for lyophilisation such as water, acetic acid, or 1,4-dioxane, or mixtures thereof. Most preferably acetic acid is used because of its excellent solubilisation of fibrillary materials. The provided suspension has preferably been at rest for at least an hour, more preferably for at least six hours. After casting the suspension it is preferred that air bubbles be expelled, such as via tapping the container, or via heating, or via sonication. The provided suspension preferably comprises from 0.1% to 4% by weight of material, more preferably from 0.5% to 2%, most preferably from 0.75% to 1.75%.

It was found that a higher wt.-% of material led to a slower rate of expansion. For instance, a plug formed from a 1.5 wt.-% collagen suspension was found to have a slower rate of expansion than a plug formed from a 1.0 wt.-% suspension. Accordingly, for a plug with a faster rate of expansion, the provided suspension preferably comprises from 0.1% to 2% by weight of material, more preferably from 0.5% to 1.5%, most preferably from 0.6% to 1.0%. For a plug with a slower rate of expansion, the provided suspension preferably comprises from 1% to 6% by weight of material, more preferably from 1.25% to 4.0%, most preferably from 1.5% to 2.0%.

In some embodiments, the invention relates to a method of manufacturing an expandable plug as described herein, comprising:
providing a suspension or solution of a material for forming the plug;
homogenizing the suspension or solution;
casting the suspension or solution;
freezing the suspension or solution;
optionally lyophilizing the suspension or solution;
crosslinking the optionally lyophilized solids;
optionally, performing one or more washing steps; and
optionally, lyophilizing a second time,
   to form an expandable plug wherein the material forming the plug is crosslinked as described herein. Washing steps may be included to stop the crosslinking process and wash away side-products. Washing is preferably performed using water or aqueous buffers. The optional crosslinking step may be based on any of the crosslinking methods described elsewhere herein.

### General Information

For any embodiment herein, whenever dimensions are discussed, the dimensions refer to the expandable plug in its original shape and condition, before any compression or crimping and subsequent expansion have taken place.

Unless stated otherwise, all technical and scientific terms used herein have the same meaning as customarily and ordinarily understood by a person of ordinary skill in the art to which this invention belongs, and read in view of this disclosure.

In this document and in its claims, the verb “to comprise” and its conjugations is used in its nonlimiting sense to mean that items following the word are included, but items not specifically mentioned are not excluded. In addition, the verb “to consist” may be replaced by “to consist essentially of” meaning that a composition as described herein may comprise additional component(s) than the ones specifically identified, said additional component(s) not altering the unique characteristic of the invention. In addition, the verb “to consist” may be replaced by “to consist essentially of” meaning that a method as described herein may comprise additional step(s) than the ones specifically identified, said additional step(s) not altering the unique characteristic of the invention.

Reference to an element by the indefinite article “a” or “an” does not exclude the possibility that more than one of the element is present, unless the context clearly requires that there be one and only one of the elements. The indefinite article “a” or “an” thus usually means “at least one”. As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The word “about” or “approximately” when used in association with a numerical value (e.g. about 10) preferably means that the value may be the given value (of 10) more or less 10% of the value. As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated.

All patent applications, patents, and printed publications cited herein are incorporated herein by reference in their entireties. One skilled in the art will recognize many methods and materials similar or equivalent to those described herein, which could be used in the practice of the present invention. Indeed, the present invention is in no way limited to the methods and materials described.

The present invention is further described by the following examples which should not be construed as limiting the scope of the invention.

### DESCRIPTION OF THE FIGURES

FIG. 1 Mean diameter of crosslinked plugs as compared to untreated plugs after swelling in PBS for various times. The crosslinked plugs returned to their initial diameter after crosslinking, whereas the untreated plugs did not, or at least not within a reasonable time frame. The reference lines indicate the mean diameter of the dry plug before crimping in both conditions. Results represent the mean±standard deviation.

FIG. 2 Expandable collagen plug applied in human fetal membranes ex vivo. Human fetal membranes were filled with water (left) and 1% Azure A solution (right) to show the efficacy of the 1% type I collagen plug. No leakage was observed when the plug was placed in the punctured membranes. Arrowhead indicates the plug inside the fetal membranes.

FIG. 3 Expandable collagen plug applied in a high-pressure model. A porcine urinary bladder was filled with 0.1% (w/v) 1,9-methylmethylene blue in PBS and punctured by a fetoscopic instrument (black arrowhead), through which the collagen plug was placed. The collagen plug turned blue by the 1,9-methylmethylene blue, but no fluid leaked out of the plug (white arrowhead). Without the collagen plug, the fluid squirted from the puncture site (white asterisk).

FIG. 4 Representative images of various plugs. Plugs shown are all made from collagen, and are not crosslinked (top), crosslinked (middle), and compressed after crosslinking (bottom).

FIG. 5A An expandable plug as used in FIG. 1 was placed in a transparent plastic membrane to visualise the internal expansion where the plug is surrounded by polar liquid (here PBS). FIG. 5B Additional view as for FIG. 5A.

FIG. 6 Time lapse photos of an expandable plug as used in FIG. 2 placed in a polar liquid (here PBS) at 0, 2, 4, 6 and 8 s after wetting.

FIG. 7 Time lapse photos of the recovery after deformation of a fully expanded plug (according to the invention) in polar liquid (here PBS) from 0-6 s after deformation.

FIG. 8 Time lapse photos of the recovery after deformation of a fully expanded plug (reference plug from example 5) in polar liquid (here PBS) from 0-5 min after deformation.

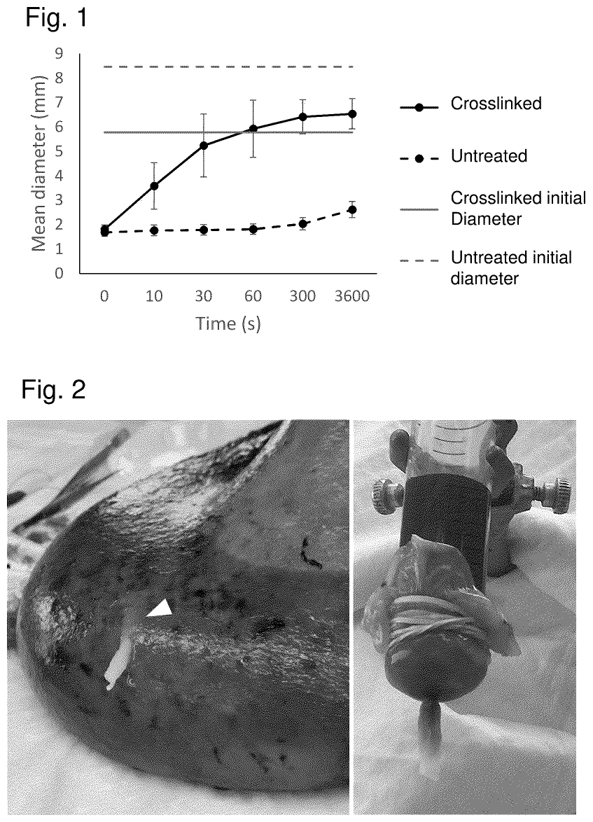

### EXAMPLES

### Example 1

For sealing of fetal membrane defects after a fetoscopic surgery, an expandable type I collagen plug is made. This plug fits through the fetoscopic instruments and directly expands when coming into contact with the amniotic fluid to seal the defect and fixate itself.

Type I collagen was isolated from bovine tendons by pulverising and a purification process including several washing steps with aqueous solutions of NaCl, urea, acetic acid, acetone and demineralized water. Purified type I collagen fibrils were suspended in 0.25 M acetic acid to a 1.5% (w/v) collagen suspension and swollen overnight. The suspension was homogenized and cast in a tube with an inner diameter of 9.9 mm, while reducing the presence of air bubbles. The filled tubes were frozen at −20° C. and lyophilised. Lyophilised plugs were crosslinked using a zero-length crosslinking method applying 1-ethyl-3-(3-dimethylaminopropyl)carbodiimide (EDC) and N-hydroxysuccinimide (NHS). In short, the plugs were wetted overnight in 50 mM 2-morpholinoethane sulfonic acid (MES buffer, pH 5.0) containing 40% (v/v) ethanol, followed by crosslinking for 3 h at ambient temperature in 33 mM EDC and 6 mM NHS in 50 mM MES buffer (pH 5.0), containing 40% (v/v) ethanol, and washed with 0.1 M Na₂HPO₄, 1 M NaCl, 2 M NaCl, and demineralized water, after which plugs were lyophilised. As a final step, plugs were cut to a length of 4.5 cm and shrunken for 3×30 s at 80 psi using a Model RVJ Pneumatically-Actuated Crimping Machine (Blockwise, Tempe, AZ, USA) to fit the endoscopic instruments.

For the expansion, crimped plugs were wetted in phosphate buffered saline (PBS) and the diameter measured using a calliper at 0 s, 10 s, 30 s, 1 min, 5 min and 60 min after wetting. The crosslinked plug immediately started expanding after placing in PBS, while the untreated plugs remained small over time (Table 1). The diameter of the crosslinked plug tripled within 1 min and has more than tripled within 1 h. Here, the plugs even exceeded the diameter of the plugs before crimping.

The crosslinked collagen plug was passed through the fetoscopic instrument and expanded immediately after placement in PBS with the potential to close a defect in the fetal membranes with a diameter of more than 3 mm within 10 s. The untreated collagen plug did slightly expand in 1 h, although it did not reach the diameter of 3 mm, making it unable to fully close the fetoscopic entry point. Next to the diameter in expanded form, the time to expand is important to efficiently close the defect during surgery, which makes the untreated collagen plug unusable for this application. It was found that a higher wt.-% of material led to a slower rate of expansion.

TABLE 1
Mean diameter of crosslinked collagen plugs vs. untreated plugs after swelling in PBS at various time
points (mm ± SD). The crosslinked plugs returned to more than their initial diameter after crosslinking,
whereas the untreated plugs did not swell enough in 1 h to seal the fetoscopic entry point of 3 mm.

| | Before | | | | | | |
|---|---|---|---|---|---|---|---|
| Type plug | crimping | 0 s | 10 s | 30 s | 60 s | 300 s | 3600 s |
| Crosslinked 1.5% type | 5.78 ± 0.67 | 1.81 ± 0.18 | 3.59 ± 0.95 | 5.24 ± 1.29 | 5.93 ± 1.17 | 6.42 ± 0.70 | 6.54 ± 0.62 |
| I collagen plug | | | | | | | |
| Untreated 1.5% type | 8.47 ± 0.29 | 1.69 ± 0.16 | 1.77 ± 0.22 | 1.79 ± 0.21 | 1.82 ± 0.21 | 2.04 ± 0.25 | 2.62 ± 0.33 |
| I collagen plug | | | | | | | |

### Example 2

The efficacy of the expandable collagen plug for the use in fetoscopy is shown in an ex vivo setup with human fetal membranes. The human fetal membranes, chorion and amnion together, were obtained from the Radboud university medical center after births. The production of the type I collagen plug was almost the same as in example 1, although, here, a 1.0% (w/v) type I collagen suspension was used for the preparation of the plugs, instead of the 1.5% collagen.

Two setups were used to show the efficacy to seal the defect and prevent it from leakage. In the first setup, a sac was formed from the membranes and filled with water. As a second setup, a piece of the fetal membranes was tightened around a plastic cylinder using a rubber band. The cylinder was filled with 1% (w/v) Azure A in demineralised water to visualise the fluid uptake by the plug. In both setups, a fetoscope with a diameter of 10 Fr (3.3 mm) was used to puncture the membranes and a crosslinked plug was inserted into the defect through the sheath.

In both models, the plug started expanding when placed in the defect and no leakage was observed afterwards. In the first setup, the plug can be seen inside the fetal membranes (FIG. 2, white arrowhead). The second setup visualised the small amount of 1% (w/v) Azure A solution that was absorbed by the plug. After 1 h the plug was only partly blue and was not dripping any fluid indicating a sealed defect. The crosslinked collagen plugs were able to close the defect in the punctured human fetal membranes ex vivo and prevent it from leakage.

### Example 3

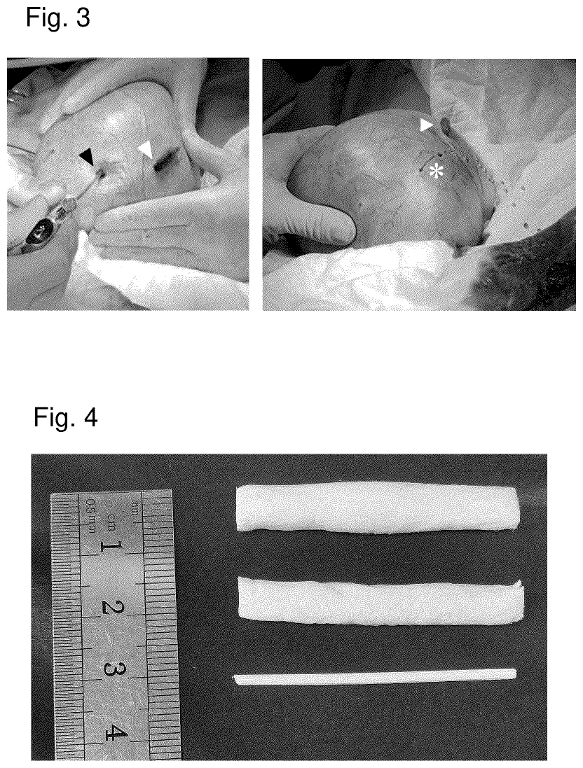

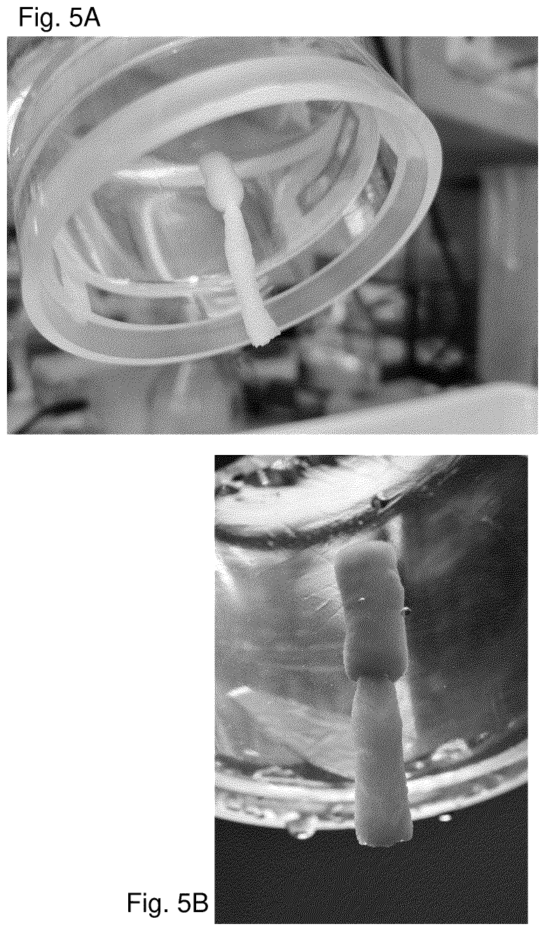

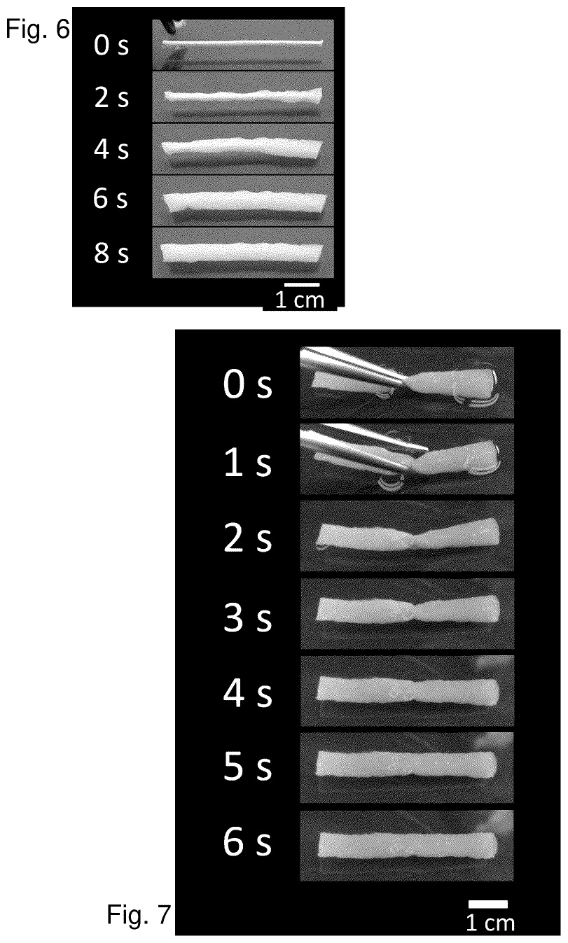

The expandable collagen plug was tested in a high-pressure model to study the efficacy of sealing the defect and fixating itself. Here, a crosslinked 1.0% (w/v) type I collagen plug was prepared as described in example 1. As high-pressure model, a porcine urinary bladder was partly exposed and filled with 0.1% (w/v) 1,9-dimethyl-methylene blue in PBS. The bladder was punctured by a fetoscopic instrument, through which the crosslinked 1% (w/v) collagen plug was placed. As a negative control, another defect was made in the bladder using the fetoscopic instrument, which was left open (FIG. 3, black arrowhead).

After placement in the defect, the expandable plug immediately started expanding, fixated itself and sealed the defect (FIG. 3, white arrowhead). The plug became wetted with 0.1% (w/v) 1,9-dimethyl-methylene blue in PBS, although no fluid did leak out of the plug. Without the collagen plug the blue fluid squirted from the puncture site (FIG. 3, white asterisk).

The high-pressure model showed that the expandable collagen plug was able to seal the defect and fixate itself despite the higher pressure inside the bladder.

### Example 4

Next to non-medical grade type I collagen, medical grade type I collagen from Southern Lights Biomaterials (now part of Collagen Solutions, London, UK) was used to compare the effect on the expansion of the plugs when placed in PBS. The plugs were made with the same procedure as described in example 1.

After crosslinking and crimping, the plugs were placed in PBS and the diameter was measured over time using a calliper. For comparison, non-crosslinked plugs from both batches were also included.

As shown in table 2, there was no difference found in swelling behaviour of the expandable plug made of the non-medical grade collagen compared to the medical grade collagen. In the non-crosslinked plug only a minor difference between both batches was found in the diameter after 300 s and 3600 s, although in both cases the non-crosslinked plug did not swell enough to seal the fetoscopic entry point of 10 Fr (3.3 mm). Overall, no relevant difference in swelling behaviour of the expandable collagen plug was found between the plugs made of medical grade and non-medical grade collagen.

TABLE 2
Mean diameter of both crosslinked and untreated plugs made of medical grade collagen
vs. non-medical grade collagen after swelling in PBS at various time points (mm ±
SD). No significant difference was found in swelling behaviour of the expandable plug
made of the non-medical grade collagen compared to the medical grade collagen. In the
untreated plug only a minor difference was found in the diameter after 300 s and 3600 s.

| | Before | | | | | | |
|---|---|---|---|---|---|---|---|
| Type plug | crimping | 0 s | 10 s | 30 s | 60 s | 300 s | 3600 s |
| Crosslinked | 5.9 ± 0.5 | 1.8 ± 0.1 | 3.7 ± 1.1 | 5.6 ± 1.8 | 6.2 ± 1.7 | 6.7 ± 1.0 | 7.0 ± 0.7 |
| non-medical | | | | | | | |
| Crosslinked | 6.0 ± 0.5 | 1.8 ± 0.2 | 4.4 ± 1.4 | 5.9 ± 1.3 | 6.3 ± 1.0 | 6.6 ± 0.7 | 6.7 ± 0.6 |
| medical | | | | | | | |
| Non-crosslinked | 8.2 ± 0.3 | 1.6 ± 0.1 | 2.0 ± 0.2 | 2.1 ± 0.2 | 2.1 ± 0.2 | 2.2 ± 0.2 | 2.6 ± 0.2 |
| non-medical | | | | | | | |
| Non-crosslinked | 8.3 ± 0.3 | 1.7 ± 0.1 | 1.9 ± 0.0 | 1.9 ± 0.1 | 2.1 ± 0.3 | 2.6 ± 0.2 | 3.0 ± 0.2 |
| medical | | | | | | | |

The crosslinked plugs from both non-medical grade collagen as well as medical grade collagen can fit through fetoscopic instruments and seal defects in fetal membranes after fetoscopy.

### Example 5

This example compares the expandable collagen plugs with shape-memory of the present invention with other types of plugs (see US 2009/0326577). The other types of plugs underwent alkaline denaturation treatment during production. Initially for both types, pulverised bovine tendon resulting in a large surface area was used as starting material to maximise the purification of the collagen. For the plugs with the alkaline treatment, 100 g pulverised tendon was washed for about 2.5 h in 333 ml 0.2% (v/v) peracetic acid in a 5% (v/v) aqueous ethanol solution with agitation. The material was rinsed 4 times with demineralised water and NaOH was added to obtain a 3 M NaOH suspension which was incubated at 37° C. for 2 h with agitation. After 2 h the pulverised tendon was fully dissolved, resulting in a complete loss of material and making it impossible to proceed with the protocol.

As pulverised tendon could not be used for the protocol of US 2009/0326577, bovine tendon was obtained, cleaned and cut in to slices of 3 to 4 mm thickness. In total 25 g of the tendon slices were washed for about 2.5 h in 83 ml 0.2% (v/v) peracetic acid in a 5% (v/v) aqueous ethanol solution with agitation. The material was rinsed 4 times with demineralised water and soaked in 83 ml 3 M NaOH solution at 37° C. for 2 h with agitation. The material was removed and rinsed in 83 ml ultrapure water for 15 min, after which 250 ml 0.2 M acetic acid was added. After 15 min with agitation, it was washed 5 times for 5 min with 83 ml ultrapure water and mechanically agitated using a polytron. As the suspension was too viscous to homogenise, 25 ml ultrapure water was added. In the end the suspension was casted in the same cylindrical moulds as used in example 1 and frozen overnight at −80° C. The frozen samples were lyophilized and crimped in the same way as the other plugs, before the expansion in both ultrapure water and PBS was examined. For this, the diameter was measured over time using a calliper at 0 s, 10 s, 30 s, 1 min, and 5 min after wetting.

TABLE 3
Mean diameter of plugs with alkaline treatment compared to crosslinked collagen
plugs after swelling in PBS or ultrapure water at various time points (mm ± SD).

| Type plug | 0 s | 10 s | 30 s | 60 s | 300 s |
|---|---|---|---|---|---|
| Bovine tendon plugs with | 2.41 ± 0.06 | 3.30 ± 1.32 | 2.71 ± 0.13 | 2.74 ± 0.19 | 2.88 ± 0.17 |
| alkaline treatment in PBS | | | | | |
| Expandable 1.35% type I | 1.70 ± 0.12 | 5.39 ± 1.03 | 6.61 ± 0.59 | 7.00 ± 0.37 | 6.85 ± 0.40 |
| collagen plug in PBS | | | | | |
| Bovine tendon plugs with | 2.47 ± 0.02 | 2.53 ± 0.07 | 2.62 ± 0.09 | 2.78 ± 0.13 | 3.16 ± 0.18 |
| alkaline treatment in | | | | | |
| ultrapure water | | | | | |
| Expandable 1.35% type I | 1.67 ± 0.10 | 5.42 ± 0.96 | 6.81 ± 0.66 | 7.15 ± 0.65 | 7.26 ± 0.43 |
| collagen plug in ultrapure | | | | | |
| water | | | | | |

The expansion of the bovine tendon plug with alkaline treatment is lower than the expansion of the crosslinked collagen plugs. The diameter of the crosslinked collagen plugs increased with about 300% within 30 s, while the diameter plugs with the alkaline treatment only increased with about 10% in 30 s. This indicates that plugs of the invention exhibit faster and larger expansion after wetting in PBS or ultrapure water as compared to plugs that underwent alkaline treatment. Plugs of the invention tripled in diameter within 10 s, exceeding the endoscopic entry point (commonly 10 Fr, 3.3 mm), and are thereby able to seal the defect directly after application. The plugs with the alkaline treatment did not exceed the 3.3 mm and would not be able to seal an endoscopic entry point. Next to the difference in expansion of both types of plugs, the use of slices of bovine tendon instead of pulverised material may result in a less pure material, making it less ideal for clinical use.

### Example 6

The shape memory in the previous examples is based on the use of zero-length crosslinking. Here, the amine and carboxylic group from the collagen molecules are crosslinked, although it is possible to add compounds that function as a crosslinking bridge or spacer. The use of such a compound could alter several properties of the collagen scaffold, such as biodegradability. In this example the effect of an additional spacer molecule on the shape memory was studied. For this, 1.35% (w/v) collagen plugs were prepared according to example 1 with the addition of 2.5 μM or 1 mM adipic acid or 2.5 μM or 1 mM hexamethylene diamine to the MES-buffer. After crosslinking and crimping, plugs were placed in PBS and the diameter was measured over time using a calliper at 0 s, 10 s, 30 s, 1 min, 5 min, 10 min and 60 min after wetting. The mean diameters of the plugs were compared with the crosslinked plugs without spacer, non-crosslinked plugs and untreated plugs from example 5.

TABLE 5
Mean diameter of collagen plugs, which are crosslinked in the presence of adipic acid
or hexamethylene diamine, after swelling in PBS at various time points (mm ± SD).

| Type plug | 0 s | 10 s | 30 s | 60 s | 300 s | 600 s | 3600 s |
|---|---|---|---|---|---|---|---|
| Plugs crosslinked in the | 1.92 ± 0.15 | 5.05 ± 0.84 | 6.66 ± 0.50 | 6.94 ± 0.56 | 7.04 ± 0.35 | 7.17 ± 0.25 | 7.33 ± 0.33 |
| presence of 2.5 μM | | | | | | | |
| adipic acid | | | | | | | |
| Plugs crosslinked in the | 1.90 ± 0.12 | 4.21 ± 0.66 | 6.09 ± 0.54 | 6.32 ± 0.43 | 6.34 ± 0.49 | 6.32 ± 0.40 | 6.80 ± 0.43 |
| presence of 1 mM adipic | | | | | | | |
| acid | | | | | | | |
| Plugs crosslinked in the | 1.98 ± 0.11 | 5.47 ± 0.82 | 6.73 ± 0.33 | 6.80 ± 0.17 | 6.72 ± 0.37 | 6.90 ± 0.32 | 7.23 ± 0.32 |
| presence of 2.5 μM | | | | | | | |
| hexamethylene diamine | | | | | | | |
| Plugs crosslinked in the | 1.99 ± 0.13 | 4.71 ± 1.04 | 6.22 ± 0.33 | 6.32 ± 0.37 | 5.91 ± 0.53 | 6.21 ± 0.37 | 6.44 ± 0.42 |
| presence of 1 mM | | | | | | | |
| hexamethylene diamine | | | | | | | |

The addition of 2.5 μM adipic acid or hexamethylene diamine to the MES-buffer during crosslinking with EDC and NHS did not result in a difference in expansion compared to the crosslinked plugs from example 5 without any additional spacer. The addition of 1 mM adipic acid or hexamethylene diamine to the MES-buffer slightly lowered the expansion.

This example shows that the shape memory can be given to collagen plugs with the addition of a spacer instead of using a zero-length crosslinker only. In high concentrations an additional spacer may alter the effect of shape memory, although, in all tested conditions, the plugs showed a fast expansion after wetting. Therefore, it may be possible to add a spacer during crosslinking to alter other properties of the expandable plug, such as biodegradability. In addition to spacers, this example makes it plausible that also other compounds can be added to the collagen plug, such as glycosaminoglycans, growth factors and pharmaceuticals, to have additional effects without interfering with the shape memory of the expandable plug. When an additional compound has an effect on the rate of expansion, it may also be used to regulate the expansion.

### Example 7

In clinical conditions the plug may deform multiple times after full expansions. Therefore, it is important for the plug to be able to adjust its shape to the changing surroundings. In this example the ability of an expanded collagen plug to return to its initial shape after deformation is tested. For this, plugs from examples 5 and 6 were pinched in the centre of the plug after full expansion with the same force using a Kocher surgical clamp, while kept in PBS or ultrapure water. The deformation was checked visually for 1 h to see if the plug returned to its fully expanded state.

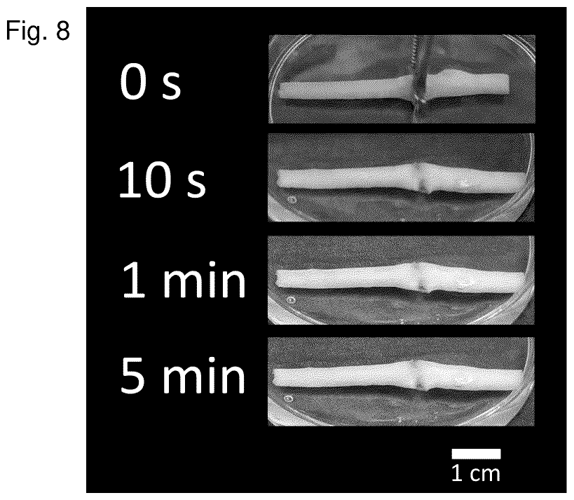

All crosslinked plugs, with or without the addition of adipic acid or hexamethylene diamine, returned to their most expanded state within 10 s and did not leave any visible damage (FIG. 7). Also repeated deformations (10 to 20) at the same point or a longitudinal deformations did not result in a permanent deformation. Macroscopically no differences were visible between the crosslinked plugs with or without the addition of adipic acid or hexamethylene diamine. In the non-crosslinked plugs the deformation did not recover within 1 h, but at the macroscopic level the plug was only deformed and not damaged. This was the same for the untreated plug in PBS, although the untreated plugs in ultrapure water were more fragile and did damage upon deformation. The plugs from example 5, which were produced using the alkaline treatment, did not recover from the deformation and showed a permanent damage at the site of deformation (FIG. 8). This result was also obtained in ultrapure water instead of PBS.

Next to the solid plugs, a sheet of Biodesign® 4-Layer Tissue Graft (Cook Medical), made of small intestinal submucosa (SIS), was rolled manually and inserted in a plastic membrane using a 10 Fr endoscope. After wetting in PBS, the sheet did hardly swell over time. Also, a part of a Biodesign® Fistula Plug (Cook Medical) was placed in PBS, but also this rolled piece of SIS did not expand over time in PBS. Without any swelling the rolled sheets may fall out of the defect or may not be able to seal the defect as it is not a solid plug. In addition, larger plugs that would be able to fill the defect may not be mounted in the application scope. Both types of rolled sheets of SIS were pinched in the same way as the solid plugs using a Kocher surgical clamp and only partly returned to their initial shape before the deformation. Minor deformations were still visible after 1 h.

Overall, the plugs according to the invention showed to be expandable plugs that are able to expand repeatedly after deformation. Other solid plugs and rolled sheets did not fully recover within 1 h after deformation. This indicates that a solid crosslinked collagen plug can react faster to deformations to maintain defect sealing.

1. An expandable plug for closing a defect in an organ or tissue without the need for sutures, wherein the material forming the plug has shape memory.

2. The expandable plug according to claim 1, wherein the material forming the plug is biodegradable.

3. The expandable plug according to claim 1, wherein the material forming the plug comprises or consists of a fibrillary material, such as collagen, such as type I collagen.

4. The expandable plug according to claim 1, wherein the material forming the plug is crosslinked.

5. The expandable plug according to claim 4, wherein the crosslinking is via the formation of covalent bonds between functional groups present in the material forming the plug.

6. The expandable plug according to claim 1, wherein the plug has homogenous density and content throughout the plug.

7. The expandable plug according to claim 1, wherein the defect is in a membrane, such as a fetal membrane.

8. The expandable plug according to claim 1, wherein the defect is an endoscopic entry point.

9. The expandable plug according to claim 8, wherein the defect is an endoscopic entry point that was created during fetal surgery.

10. The expandable plug according to claim 1, wherein it has a length of at most about 10 cm along its longest axis, and/or wherein it has a length of at most about 1 cm along its shortest axis, preferably wherein the expandable plug has one longest axis and two substantially identical shorter axes.

11. The expandable plug according to claim 1, wherein the material forming the plug is native collagen.

12. The expandable plug according to claim 11, wherein the material forming the plug comprises or consists of a fibrillary material, such as collagen, such as type I collagen.

13. The expandable plug according to claim 12, wherein the material forming the plug is crosslinked.

14.-18. (canceled)

19. Method for producing a plug according to claim 1, wherein the method comprises the steps of:
a) Providing a collagen source;
b) Purifying collagen from the source to obtain native collagen;
c) Swelling the native collagen in a suitable liquid such as acetic acid to obtain swollen collagen;
d) Casting the swollen collagen in a mould to obtain cast collagen and lyophilizing the cast collagen to form an expandable plug;
e) Optionally crosslinking the expandable plug.

20. The method of claim 19, further comprising the step of:
f) lyophilizing the crosslinked expandable plug.

21. The method according to claim 19, wherein the collagen source is animal tendons.

22. The method according to claim 19, wherein the purification to obtain native collagen is without exposing the collagen to an alkaline denaturation treatment.

23. A method for closing a defect in an organ or tissue without the need for sutures, the method comprising introducing the expandable plug into the defect.

24. The method according to claim 21, wherein the defect is an endoscopic entry point, preferably an endoscopic entry point that was created during fetal surgery.

25. The plug according to claim 1, wherein the plug is an endoscopic device.
